(19) United States
(12) Patent Application Publication
(10) Pub. No.: US 2016/0296200 A1
(43) Pub. Date: Oct. 13, 2016

(54) MD (The Master Diagnostician)

(71) Applicant: Jose Luis Hinojosa, Johnson, KS (US)

(72) Inventor: Jose Luis Hinojosa, Johnson, KS (US)

(21) Appl. No.: 14/887,105

(22) Filed: Oct. 19, 2015

Related U.S. Application Data
(60) Provisional application No. 62/103,705, filed on Jan. 15, 2015.

(51) Int. Cl. A61B 7/04; G06F 3/0484; G06F 3/0488; H04N 5/77; A61B 6/00; A61B 5/0205; A61B 5/00; A61B 7/00; A61B 8/08; G06F 3/0482; A61B 5/1455

(52) U.S. Cl. CPC A61B 7/04; G06F 3/0482; G06F 3/04847; G06F 3/0488; H04N 5/77; A61B 5/14551; A61B 5/0205; A61B 5/742; A61B 5/7475; A61B 7/003; A61B 8/0883; A61B 8/085; A61B 6/461; A61B 6/467; A61B 5/0077

(57) ABSTRACT

This document presents a system and method for The Master Diagnostician (The MD) an electronic stethoscope having additional features not found in a typical electronic stethoscope. The additional features include sensors and modalities for sound capture, pulse oximetry, heart sound and rhythm interpretation, ultrasound capability, and an integrated multi-megapixel camera to record still images and/or video data. The MD also records and stores ultrasound, camera, pulse oximetry, and sound data for later recall and transmission to external devices either through wired or wireless data communication and may he used for TeleMedicine consultations and referrals to medical practitioners located at different sites.

### PRIORITY

This application claims priority to provisional patent application 62/103,705, filed Jan. 15, 2015, entitled “The MD (The Master Diagnostician)”, which is included herein in its entirety by reference.

### COPYRIGHT NOTICE

A portion of the disclosure of this patent document contains material which is subject to copyright protection. The copyright owner has no objection to the facsimile reproduction of the patent document or the patent disclosure, as it appears in the

Patent and Trademark Office patent file or records, but otherwise reserves all copyright rights whatsoever.

### BACKGROUND

Medical devices continue to evolve to meet the needs of physicians and other medical care providers to diagnose and resolve medical issues of patients in their care. A medical device that can serve as both a diagnostic tool and a teaching tool is preferred by medical professionals as such devices can serve multiple functions without the need for additional, and perhaps costly, separate devices. Another consideration in the medical field is the need to improve upon diagnostic and teaching tools from the devices that have been used in the past. A device that was adequate for recording certain patient parameters 20 or 30 years ago may be entirely inadequate for the current state of medical practice.

Additionally, improving the speed of the intake of diagnostic information is a goal of improvements to existing medical devices. The more quickly a medical professional can record and access information about a patient, the more quickly a medical diagnosis or instruction point may be made and the more quickly a patient's needs may be served. One means for increasing the speed of medical data intake is to create new medical devices that are capable of measuring and capturing multiple physical parameters of a patient. A medical device that may measure and record several important diagnostic data points from a patient permits a medical professional to perform patient parameter intake without having to switch from one medical device to another. This results in time savings for the medical professional and a quicker pathway to helping patients or instructing students in a teaching environment.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a proximate upper surface view of The MD device consistent with certain embodiments of the present invention.

FIG. 2 is a distal upper surface view of The MD device consistent with certain embodiments of the present invention.

FIG. 3 is a view of a view of the ultrasound probe attached to The MD device consistent with certain embodiments of the present invention.

FIG. 4 is a view of bottom and left side portions of The MD device consistent with certain embodiments of the present invention.

FIG. 5 is a view of bottom and right side portions of The MD device consistent with certain embodiments of the present invention.

FIG. 6 is an operational module component diagram for The MD device system components consistent with certain embodiments of the present invention.

FIG. 7 is an operational flow diagram for The MD device operational modalities consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Reference throughout this document to “mobile device” refers to any handheld device such as, but not limited to, a smart phone, tablet, iPad, network computer, watch or any other device a user may carry when travelling from place to place and interact with one or more networks.

Electronic stethoscopes are known in the art and are in current use in the field of medicine where they are an important diagnostic tool for physicians, nurses, health care students, Emergency Medical Technicians, and other medical caregivers. A stethoscope permits a health care professional to listen to lung, abdomen, and heart sounds and determine heart rhythm. The electronic stethoscope is additionally used to provide heart rate readings and permits uploading of heart sounds into electronic medical records. A stethoscope, either conventional or electronic, may also be employed in determining a patient's blood pressure when used in conjunction with a blood pressure cuff.

As important as the current functions of electronic stethoscopes are, an electronic stethoscope is just one of a number of diagnostic tools that a health care provider must employ to provide an assessment of the condition of a patient. Additional physiological parameters, such as capturing and recording ultrasound information, or determining blood oximetry must be captured and transmitted to an electronic health record through the use of additional medical devices. This requires a health care provider to have these devices with him/her during diagnostic meetings, or have a patient in a room containing a complete array of such diagnostic equipment.

In an embodiment, the instant disclosure provides the recitation for a medical device, The Master Diagnostician device (The MD), having more features than a similar electronic stethoscope device currently in use. The MD is an electronic stethoscope that performs the same functions as electronic stethoscopes currently in use by health care professionals, but also provides additional functions which a typical electronic stethoscope is not capable of providing. The additional functions include the ability to measure pulse oximetry, interpretation of heart sounds, interpretation of heart rhythm, ultrasound, and photographs utilizing a visual range camera. Measurements of these physical parameters may be uploaded from the diagnostic medical device into each patient's electronic medical record. These measurements and records may be used to enable and enhance TeleMedicine consultations and medical referrals at sites distant from the patient's location.

In an embodiment, The MD is used by a medical professional to measure a number of physical parameters and diagnose a range of conditions. To begin, the user may insert both earpieces in the user's ears, just as in the use of a conventional electronic stethoscope, and may place the measurement portion of The MD on the area to be examined. In various non-limiting embodiments, the measurement portion may be placed against the chest, back or abdomen of a patient to take measurements of the heart, lungs, or abdomen of the patient, as well as other portions of the anatomy. If the user does not select one of the provided modalities and listens first to the heart, the heart rate only will appear on a screen built into The MD. If the user believes he/she heard an abnormal heart sound, the user may press the button associated with the interpretation of heart sounds modality on The MD, and The MD will interpret the abnormal sound and display the interpretation on the screen built into The MD. If the user wishes to save this sound, a record of the sound may be saved by pressing a “record” button built into the device and a record of this sound will be stored in the internal memory of The MD device, and may be output to a patient's electronic medical record as the sound is recorded or upon later recall from the internal memory. In an embodiment, should the medical professional wish to measure the oximetry of the patient, the medical professional may select the modality button associated with pulse oximetry to place The MD in a mode to measure this parameter. The user may place the probe section of the measurement portion of The MD on a designated area of the patient and the value of the patient's oximetry will be displayed on the integral LCD screen.

In an embodiment, to measure suspected abnormal heart rhythms, the user may select the button for the modality associated with the interpretation of heart rhythm. The user then places the bell of The MD over the patient's heart. The heart rhythm will be interpreted by The MD and displayed on the integral LCD screen. In an embodiment, to perform ultrasound measurements, the user may press the button on The MD associated with the ultrasound modality. The user may then apply the probe section of the measurement portion on the area of the patient to be examined. In non-limiting examples, the probe section could be applied to the heart area of the chest, or to the patient's abdomen, or other portions of a patient's anatomy. If the user sees a measurement on the integral LCD screen display that the user wishes to save for later review, the user may press the “record” button on The MD and capture an ultrasound video or still image that is then stored in the internal electronic memory of The MD.

In an embodiment, if a patient presents as having a rash of an unknown variety and the user is uncertain as to the nature of the rash, the user may press the button on

The MD associated with the camera modality. The user may then take a visual light photograph of the condition as a snapshot which is then stored in the internal electronic memory of The MD. Likewise, the camera modality may be used to document the healing and/or progression of skin or wound infections, and the images obtained via this modality can then be uploaded into the patient's electronic medical records.

In an embodiment, any of the information acquired during an examination of a patient may later be uploaded into each patient's electronic medical records. These records may be later accessed by the user, or may be readily accessible for TeleMedicine consults with specialists anywhere in the world.

In a non-limiting embodiment, The MD is 28 to 30 inches in length. At this size, The MD is difficult to misplace. Also, if any unauthorized individual obtains possession of a The MD device, that individual will be unable to retrieve any of the confidential information that is stored within the electronic memory of The MD.

In an alternative embodiment, the ultrasound component may be used to evaluate, for example, chest and abdomen trauma patients. Fluid collections around the heart, such as pericardial effusions, and intra-abdominal bleeding can be quickly identified. Also, the heart's ejection fraction (EF) may be measured with the ultrasound probe. Additionally, gallbladder stones may be diagnosed through the ultrasound modality, as well as the viability of an early pregnancy. In an additional alternative embodiment, the rhythm interpretation modality and components may be expanded to include a formal electrocardiogram as a future exemplary function of The MD. The MD translates into faster and more accurate diagnoses, initiation of life-saving treatments earlier in the disease process, more timely and thorough documentation, less likelihood for individuals to consider medical fraud due to the more thorough and complete documentation of any condition during consultation, more teaching opportunities, better doctor-patient relationships, and ultimately, better outcomes for each patient's health.

In an exemplary configuration, The MD may have earpieces, or eartips, a binaural eartube, flexible tubing, an LCD touch screen, buttons for a plurality of modalities. The modalities may include pulse oximetry, interpretation of heart sounds, interpretation of heart rhythm, ultrasound, and a digital camera. In this configuration, the heart rate automatically appears on the screen, without having to push a modality button. The MD may have one or more Universal Serial Bus (USB) ports. In an exemplary embodiment, The MD on-off button may be located in the middle of the device, flanked by the volume button on one side and the record/save/snapshot button on the opposite side of the on-off button. The MD may also have a diaphragm/bell of a chest piece. The MD may change the modality between a diaphragm or bell with the push of a selection button. There is a probe component of the chest piece as well to enable the ultrasound and pulse oximetry measurements to be performed.

In an exemplary embodiment, the components work together to provide measurements for each selected modality. In an exemplary configuration, two earpieces or eartips are configured for binaural use and are attached to solid, preferentially inflexible, eartubes. The inflexible eartubes are attached and connected to a flexible tube, which is then connected to the motherboard having a processor that serves as the operational primary processor of The MD. The primary processor is the approximate size of a cell phone, although this should not be considered limiting as additional reductions in the size of the primary processor may occur as technology advances, and may have an LCD touch screen.

In this exemplary embodiment, The MD has a selection device, such as a button, for each modality that may be selected for operations to be performed by The MD. Additionally, The MD has an on/off selection element, a selection element to permit turning the volume on or off, as well as selecting the volume level, The MD has a selection element to select a record/tape/ or snapshot action, and incorporates three USB ports for transferring information to electronic storage such as electronic patient health records, or electronic storage for later review, for communication in TeleMedicine consultations, and for transmission to other medical practitioners for referrals at distant sites.

The primary processor connects to the chest piece of The MD. The chest piece is the element of The MD that may come into contact with the patient during certain selected modalities of performance. The chest piece may have a bell-shaped component for low frequency listening by a medical practitioner, and a diaphragm component for capturing or measuring higher frequencies on one side of The MD central portion. On the other side of The MD central portion may be a probe to be used for modalities in which ultrasound and pulse oximetry actions are performed.

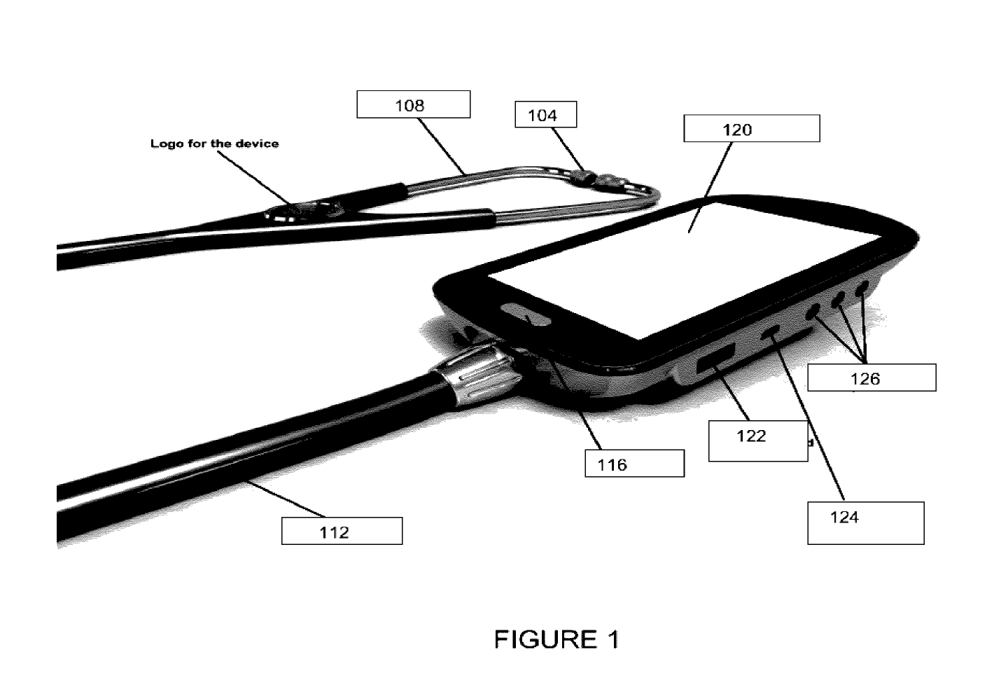

Turning now to FIG. 1, this figure presents a proximate upper surface view of The Master Diagnostician (The MD) device consistent with certain embodiments of the present invention. In an exemplary embodiment, the proximate upper surface of The MD 100 recites the portion of The MD that closest to the practitioner and provides a medical practitioner or student with a standard earpiece 104 as the transmission method for patient sound measurements from The MD when it is attached to a patient. Just as in electronic stethoscopes currently in common use, the MD has two standard earpieces 104, one earpiece for insertion into each of the ears of a medical practitioner or medical student. In an alternative embodiment, The MD may come equipped with eartips (not shown) in place of the two standard earpieces 104. The earpieces 104 or eartips (not shown) form the terminal ends of a binaural eartube 108, which serves to split sound captured by The MD and deliver the captured sound to both ears of a medical practitioner or medical student. The binaural eartube 108 joins into a single flexible tube 112 that connects the earpieces 104 to the sensor portion of The MD 114.

In an exemplary embodiment, the sensor portion 114 of The MD performs all of the measurement functions of The MD. The sensor portion 114 may have an on/off selection element 116 which serves to turn The MD off when the instrument is not in use so as to preserve battery life, and to keep unauthorized use of The MD instrumentation to a minimum. The upper surface of The MD sensor portion 114 is defined as that surface of The MD sensor portion 114 that is facing the medical practitioner or medical student when The MD is in operational use. The upper surface of The MD, as herein defined, may have a Liquid Crystal Diode (LCD) touch screen 120 as the central element of the upper surface of the sensor portion 114. The LCD touch screen permits the display of current or stored measurement data, and permits the indication of actions selected when selection options are displayed to the medical practitioner or medical student during operation. In this exemplary embodiment, a medical practitioner or medical student may have access to a Universal Serial Bus

(USB) port 122 that permits the insertion of an ultrasound probe, a mini-USB port 124 that permits the wired connection to a computer system permitting an interface with software modules on said computer system, and two or more audio output ports 126 to permit multiple listeners to access the sound measurements collected during The MD operation. The multiple audio output ports 126 are particularly advantageous to medical students who are associated with a medical practitioner, permitting the medical practitioner to listen to a sound and provide diagnostic and instruction information about the measured sound as the medical practitioner and medical students experience the measured sound in real time. The MD may also access and play back measured sounds that have been stored within the digital memory contained in the sensor portion 114 such that a medical practitioner may instruct medical students as to the diagnostic information to be gained from a measured sound as the medical practitioner and a plurality of medical students listen to the playback of the recalled sound at the same time.

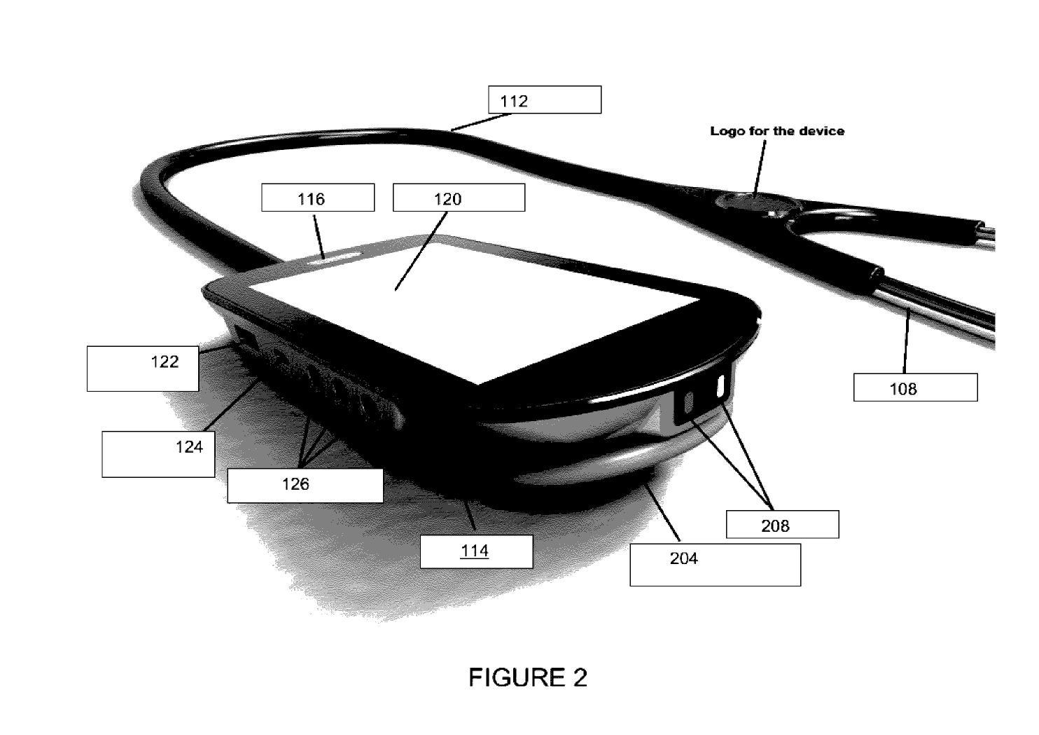

Turning now to FIG. 2, this figure presents a distal upper surface view of The MD device consistent with certain embodiments of the present invention. In an exemplary embodiment, the system presents the distal upper surface of The MD 200, which presents the portion of the upper surface of the sensor portion 114 that is closest to the patient. In this exemplary embodiment, the distal upper surface of The

MD 200 discloses the binaural eartube 108, the flexible tubing 112, the on/off selection element 116, the LCD touch screen display 120, USB port for ultrasound probe 122, mini-USB port for computer interface 124, and a plurality of audio outputs for multiple listeners 126. The distal upper surface of The MD 200 also discloses the chest piece 204 that may be composed of both a bell and diaphragm for use in contact with the patient body parts. The chest piece 204 may be placed in contact with any portion of the patient's anatomy but may be preferentially placed against the chest, stomach, back or any other portion of the patient's anatomy to record sound. The bell is preferentially utilized when low frequency sounds are to be captured, or actively listened to by one or more medical practitioners and/or medical students. The diaphragm is preferentially used when high frequency sounds are to be captured or used in conjunction with a probe to capture and store ultrasound or pulse oximetry measurements.

In this embodiment, the distal upper surface of The MD 200 also provides two pulse oximetry indicators 208 utilized in a pulse oximetry measurement. The two pulse oximetry indicators 208 present the medical practitioner and/or medical students with visual indications of when the measurement has begun and completed. The pulse oximetry measurement value is then retained in the electronic memory embedded in the sensor portion 114 of The MD device for later recall and review. Alternatively, the measurement may not be retained at the direction or discretion of the medical practitioner.

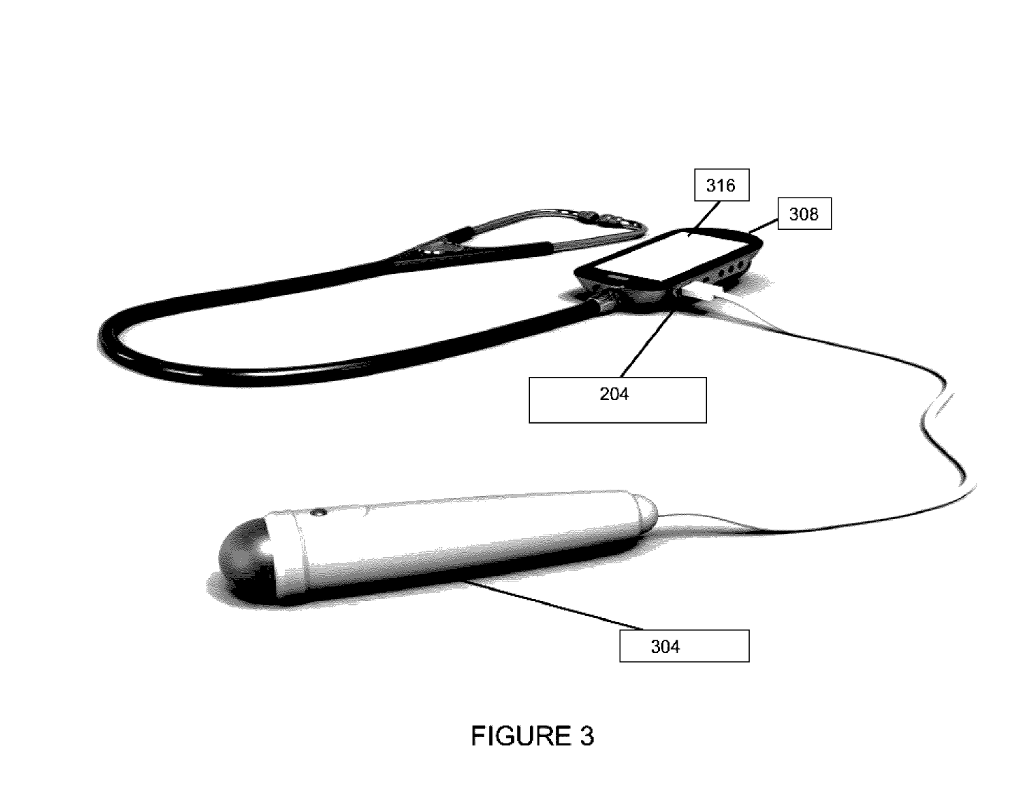

Turning now to FIG. 3, this figure presents a view of the ultrasound probe attached to The MD device consistent with certain embodiments of the present invention. In an exemplary embodiment, The MD may be connected to an ultrasound probe sensor element 304 to permit a medical practitioner to collect ultrasound images from various portions of the patient's body. The ultrasound probe sensor 304 is a well-known handheld device that may connect to The MD 308 through a USB port 312 that is normally reserved as an ultrasound probe connection, but which may, in alternative embodiments, be a connection point for other instruments and sensors that connect through a USB port 312.

The ultrasound probe sensor 304 may be configured with an internal battery to provide power to the sensor. The sensor measurements are communicated through the wired connection from the ultrasound probe sensor 304 to The MD 308 through the USB port 312 connector and stored within the internal digital memory of The MD 308. The ultrasound probe sensor 304 data may be immediately displayed on the upper surface LCD display screen 316 of The MD 308. In an additional embodiment, the ultrasound probe sensor 304 data may be recalled for later review, or snapshots and video snippets of the data may be pulled from the measurements and transmitted to additional medical practitioners for TeleMedicine consultations and referrals at distant sites.

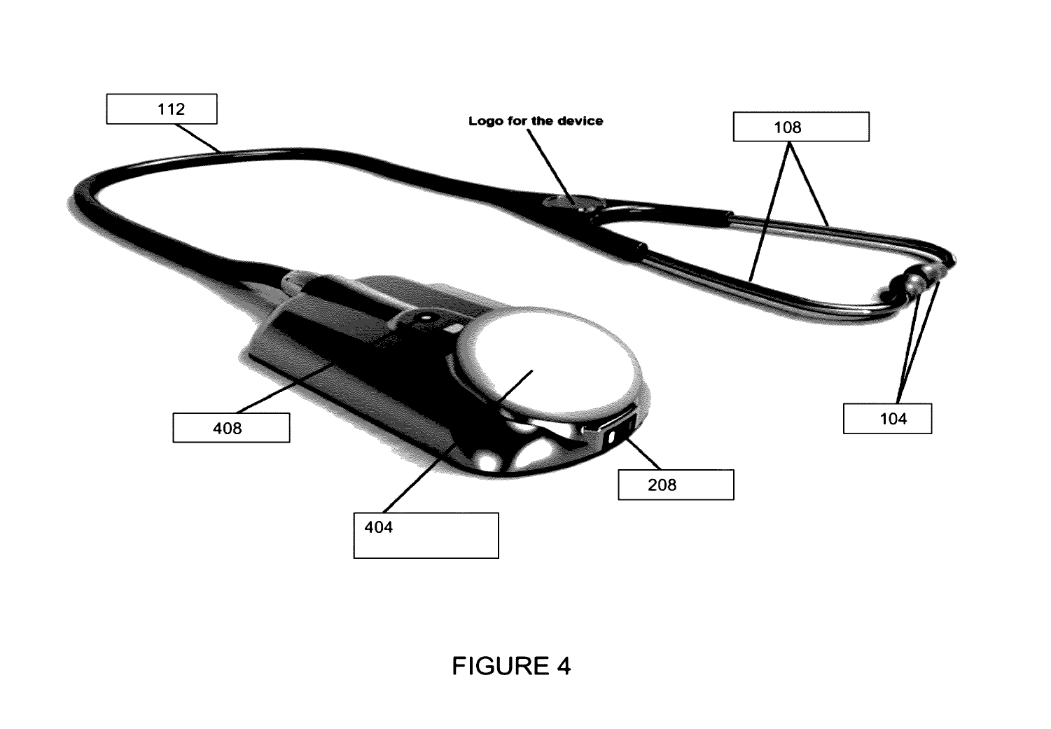

Turning now to FIG. 4, this figure presents a view of bottom and left side portions of The MD device consistent with certain embodiments of the present invention. In an exemplary embodiment, The MD incorporates sensors and instruments that support modalities of measurement capture that extend the capabilities of The MD beyond that of a typical electronic stethoscope, while maintaining form and use factors similar to typical electronic stethoscopes. As part of the sensor suite installed within The MD, the bottom surface, which is typically the patient-facing portion of The MD device, may have a bell and diaphragm 404 installed within The MD device as a portion of the chest piece of The MD device. The term “chest piece” should not be considered limiting as this portion of The MD device may be typically placed against the chest to record heart and lung sounds, however, it may also be placed upon or against other portions of the patient's body to capture additional sounds or measurements. The bell and diaphragm 404, when placed against the appropriate portion of a patient's body, provide for the capture of sound measurements for use in at least the heart rate reading, interpretation of heart sounds, and interpretation of heart rhythm modalities.

In an embodiment, upon placing the chest piece bell and diaphragm sensor of The MD against a patient's chest, The MD device automatically records the patient's heart rate and displays this measurement on the LCD screen (item 120 in FIG. 1) of The MD device. Upon measuring the patient's heart rate and heart sounds, the medical practitioner may continue to listen to the sound of the patient's heart. If the medical practitioner determines that the heart sounds seem abnormal, the medical practitioner may then select a modality for the interpretation of heart sounds by selecting the button or other selector mechanism to which the interpretation of heart sounds modality has been assigned. Upon selection of this modality, The MD may interpret the abnormal sounds and display the resulting interpretation on the LCD screen of The MD device. If the medical practitioner wishes to retain a record of the abnormal heart sounds, the medical practitioner may then select a button that initiates a record feature to store the heart sounds recording and heart sounds interpretation in the digital memory of The MD device.

In this embodiment, The MD device also incorporates a digital camera 408 on the side of The MD device that typically faces the patient. The digital camera 408 is present on the side that faces the patient to permit a medical practitioner to point the camera at an area of concern on the patient's person and be presented with a display of the camera's field of view on the LCD screen. The medical practitioner may then select the camera operation selection button to capture the snapshot of the field of view. This visual record may be saved to the digital memory of The MD device for later review, transmission to another practitioner for a medical consultations and referrals, and inclusion in the patient's electronic health care record.

This figure also presents the additional elements of The MD device, described previously, such as the earpieces 104, the binaural eartubes 108, the flexible tubing 112, and the pulse oximeter 208 that permit a medical practitioner or medical student to properly utilize The MD device.

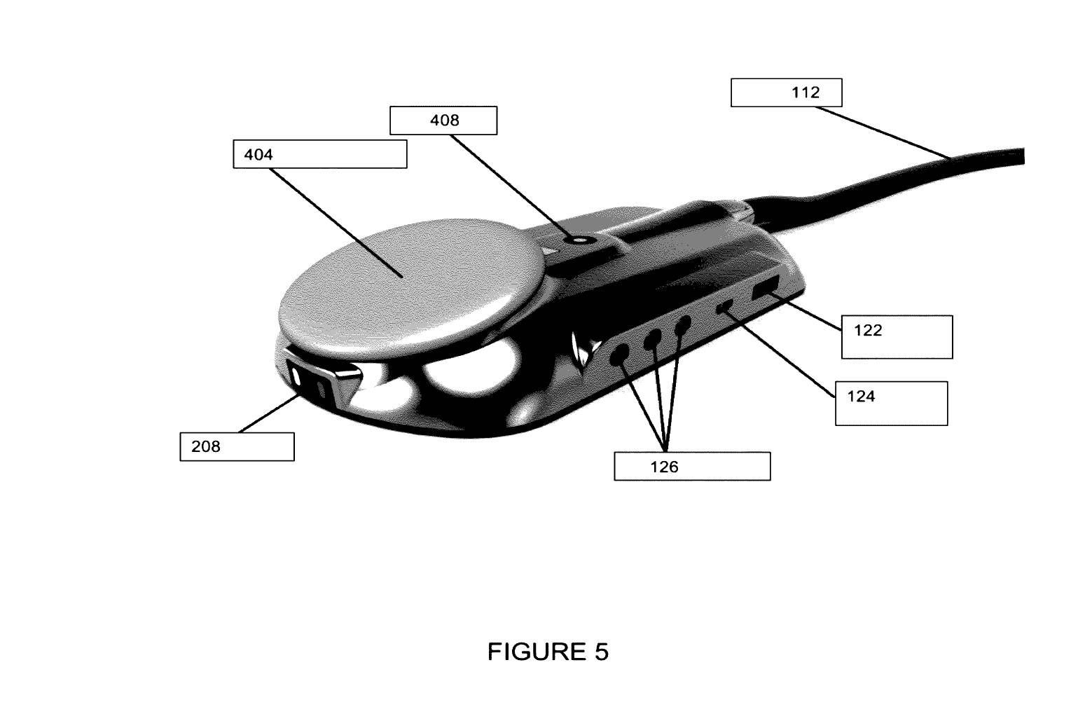

Turning now to FIG. 5, this figure presents a view of bottom and right side portions of The MD device consistent with certain embodiments of the present invention. In an exemplary embodiment, the “bottom” surface of The MD device is the surface that is typically oriented toward the patient. The bottom surface, which is typically the patient-facing portion of The MD device, may have a bell and diaphragm 404 installed within The MD device as a portion of the chest piece of The MD device. As described previously, the bell and diaphragm sensors detect and record sounds, such as heart sounds, in which the medical practitioner may have interest. In an additional embodiment, one or more students, up to the number of sound ports available on The MD device 126, may connect earbuds into The MD device sound ports 126 and listen to the heart sounds being captured by the bell and diaphragm as the medical practitioner provides commentary and other information to the students.

As previously described, but seen here in this view of the bottom and right side portions of The MD device, the medical practitioner or student may also have access to additional sensor ports for capturing additional diagnostic measurements. The MD device may have a built-in USB port 122 for attaching a separate ultrasound probe sensor, a mini-USB port for the attachment of an interface cable to a computer system 124, pulse oximeter indicators 208, and a digital camera 408 for the capture of visual information for use in diagnosis, TeleMedicine actions, and referrals. Also as previously described, the flexible tubing 112 is present in this figure to illustrate the connection between The MD device and the earpiece portions (not shown) utilized by a medical practitioner or medical student to directly access and observe the measurements made by The MD device sensors.

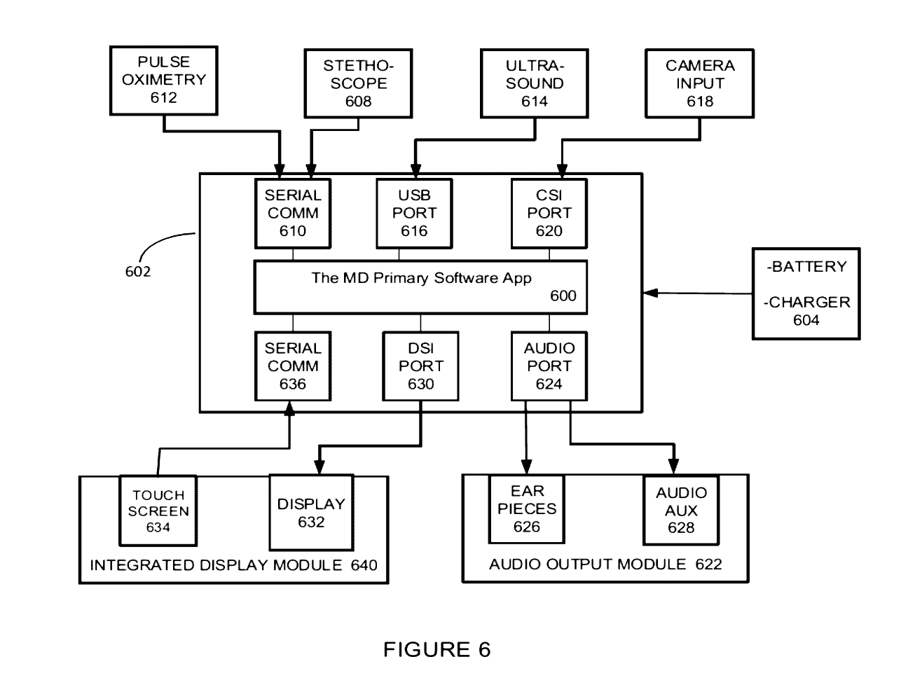

Turning now to FIG. 6, this figure presents an operational module component diagram for The MD device system components consistent with certain embodiments of the present invention. In an embodiment, the central control for The MD device and all operating modalities of which The MD device is capable is The MD Software Application (MDSA) 600. The MDSA is installed in the central processor 602 that forms the primary operational element of The MD device. The central processor provides for primary processing, memory storage, sensor interface, data capture, display screen operation, and all data output to devices used to inform medical practitioners, computers interfaced with The MD device, and data recall and transfer. The central processor 602 is powered by an internal rechargeable battery 604 that has charging circuitry attached to the rechargeable battery 604 to permit an outside source of electricity to be connected to The MD device and recharge the rechargeable battery 604 either when The MD device is in use or in hibernate mode.

In an embodiment, The MD device central processor 602 is physically connected to peripheral interface modules that provide data and electrical connectivity between The MD device and the probes and sensors that capture and transmit physiological measurement information to The MD device. The principal function of an electronic stethoscope is the capture of sounds such as heart and lung sounds. The MD device performs these functions through a stethoscope module 608. The stethoscope module 608 has a sensor, which is the bell and diaphragm element described above, that captures the sound information when the sensor is placed against the skin of the patient. The stethoscope module 608 may also have an A/D converter to convert the captured analog sound data into a digital signal representative of the analog data and transmits the converted sound data to the MDSA 600 through a serial communication port 610. The converted sound data is then stored in the digital memory (not shown) that is associated with the central processor 602. The stethoscope module is always active any time The MD device is used in diagnosis or training actions, such that sound data is always captured any time the bell and diaphragm sensor element is in use.

In this embodiment, a medical practitioner or medical student may select one of several modalities to capture physiological data for a patient. A modality that may be selected by a practitioner is the reflectance pulse oximetry modality. Upon selecting this modality, the reflectance pulse oximetry module 612 is activated by the MDSA 600. The pulse oximetry module 612 consists of the same bell and diaphragm sensor as is used by the stethoscope module 608 to capture sound data. The pulse oximetry module 612 also contains a sensor controller to regulate the sampling of sound data such that a pulse oximetry measurement is properly performed. The controller may also then communicate through the serial communication port 610 to transmit the pulse oximetry measurement data to the MDSA 600. The MDSA 600 may or may not then store the data, under the direction of the medical practitioner.

In an embodiment, a medical practitioner may select additional modalities for which diagnostic data from the patient needs to be captured. The MD device permits a medical practitioner to select an ultrasound modality and upon such selection the MDSA will activate the ultrasound module 614. To perform measurements the ultrasound module 614 will require the connection of a USB capable ultrasound probe. The ultrasound probe must be connected to the central processor 602 through the USB port connected to the USB peripheral module 616 integrated into The MD device for communication between the ultrasound probe and the MDSA 600. Upon capturing ultrasound data, the ultrasound probe transmits the data through the USB port through the USB peripheral module 616 and to the MDSA 600. The medical practitioner may choose then to save the ultrasound information by selecting a record function. The MDSA 600 may then store the data into the internal digital memory associated with the central processor 602. If the medical practitioner does not choose to “record” the ultrasound data being collected from the ultrasound probe, the data is not retained in the digital memory of The MD device.

In an embodiment, a medical practitioner may select a modality for the capture of visual data. Upon the selection of this modality, the MDSA 600 will activate a camera module 618. The camera module 618 will activate a 5 megapixel (5 MP) camera that is integrated into the exterior of the case of The MD device. The 5 MP camera may then be instructed, based upon a selection by the medical practitioner, by the Camera Serial Interface (CSI) peripheral module 620 to record a snapshot of the visual data, or may place the 5 MP camera in a video capture mode to record a longer sequence of visual data. The CSI peripheral module 620 may communicate with the MDSA 600 to transmit visual snapshot or video data to the digital memory associated with the central processor 602.

In this embodiment, each modality selected by a medical practitioner requires the presentation of the data captured by the sensors to the medical practitioner such that the medical practitioner may make decisions and perform selections based upon his/her knowledge and experience. The MDSA 600 activates the audio output module 622 each time The MD device is activated for use. The audio output module 622 accepts captured audio measurement data from the MDSA 600 as it is transmitted through the audio peripheral module 624 and presented to the medical practitioner through the main audio output element; the stethoscope earpieces 626. The audio peripheral module 624 may also transmit the audio output data to one or more auxiliary audio output ports 628 to permit one or more medical students to listen to the audio output while training and explanatory information is provided by the medical practitioner.

In this embodiment, The MD device provides visual output to the medical practitioner by transmitting visual measurement data, selection and control icons, ultrasound measurement data, and video and visual camera data through a Digital Signal Interface (DSI) peripheral module 630. The DSI peripheral module 630 formats data and drives an LCD screen display 632 for the presentation of all measurement and control data in which the medical practitioner may have interest. The LCD screen display 632 may also have a touch screen capability 634 for use in receiving selections and commands from the medical practitioner. The touch screen capability 634 communicates selections and commands from the medical practitioner and presents additional touch screen selection options from the MDSA through the serial communication interface module 636. Although The MD device provides for a separate display control module function through the DSI Interface module 630 and touch screen function through the serial communication interface module 636, the display and touch screen function are integrated into a single integrated display module 640 with which the medical practitioner will interact to view visual data of any variety and provide selections and commands through the touch of icons and menu selections provided on the LCD display 632 under the control of the MDSA 600.

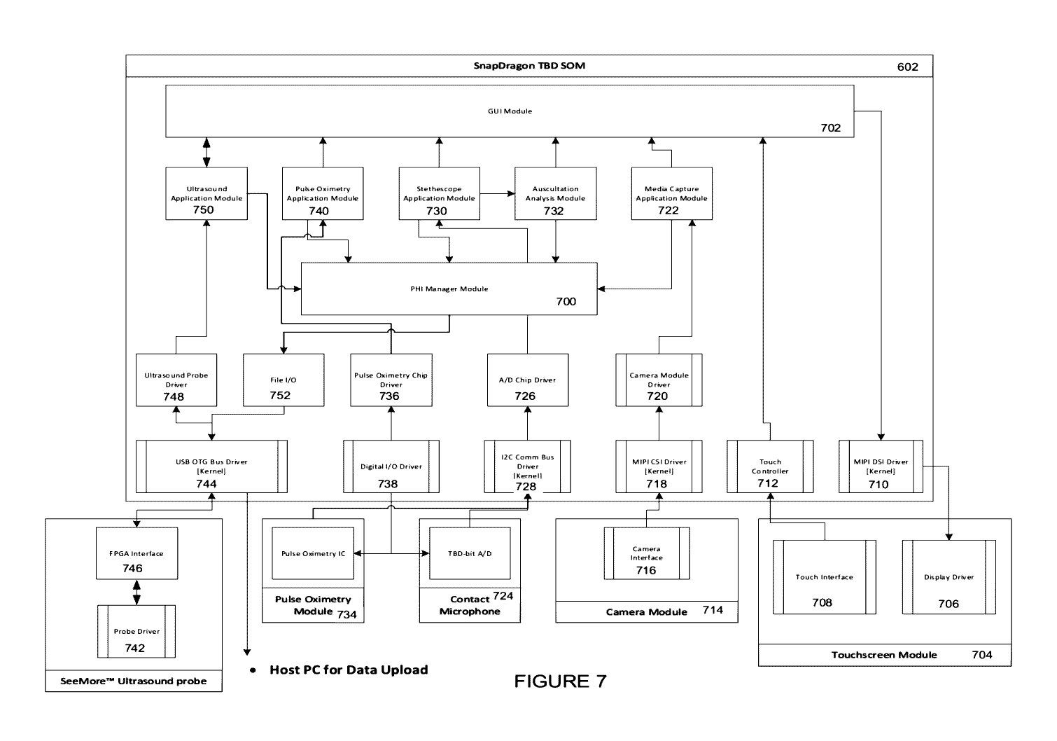

Turning now to FIG. 7, this figure presents an operational flow diagram for The MD device operational modalities consistent with certain embodiments of the present invention. In this embodiment, exemplary actions, configuration, and control features may be described that should not be considered limiting as to the features and modalities that The MD device may be capable of performing.

In an exemplary embodiment, the central processor 602 of The MD device directs all of the activities, measurements, and actions of the peripherals integrated into, and associated with, The MD device. The central processor 602 is the device within which the PHI Manager Module operating software (PHIMM) 700 is stored and active to manage all of the functions of The MD device.

In this exemplary embodiment, working in communication with the PHIMM 700 is a Graphical User Interface (GUI) module 702 which performs all of the processing and manages all of the drivers that provide information to the touchscreen module 704 for display to, and interaction with, a medical practitioner or medical student using The MD device. As previously described, the medical practitioner or medical student is presented with measurement data, ultrasound data, selection icons, selection menus, and video data on the LCD screen through a display driver module 706 and a touch interface module 708 that make up the touchscreen module 704. In an exemplary embodiment, the touchscreen module 704 is in two-way communication with the GUI module 702. The GUI module 702 transmits measurement, selection, and control information to the display driver module 706 through a DSI driver module 710 that is active to format data for display on the LCD touchscreen when transmitted to the display driver 706. The touchscreen interface module 708 is active to collect any touch control selections from the medical practitioner or medical student when control icons or menu selections are made through a touch interaction with the LCD touchscreen display. The touchscreen interface module 708 then transmits the selection and/or menu selection data to the GUI module 702 through the touch controller module 712, which formats the collected touch information into a format for use by the GUI module 702. Through this interaction, a medical practitioner or medical student is presented with all measurement and control data and permitted to interact with The MD device to direct the modalities, controls, recording, and data transfer of all measurement data collected by The MD device.

In this exemplary embodiment, The MD device permits the selection of a camera modality to permit the capture of visual data such as photographic snapshots and video imagery. The camera module 714 contains the camera interface software module 716 that controls and manages the camera device through the management software contained in the Camera Serial Interface (CSI) Driver module 718. The CSI Driver module 718 processes device data from the camera interface 716 and communicates the captured video data from the camera to the camera module driver 720. The camera module driver 720 transfers the incoming video data stream to the media capture application module 722 to perform any video manipulation to translate the incoming video data into formats that may be saved to digital database files or temporary storage files within the PHIMM 700. The media capture application module 722 also communicates incoming image data to the GUI module 702 to permit the captured video data to be processed for display on the touchscreen. The incoming video data is placed in the proper display format to permit the data imagery from the camera to be displayed on the integral LCD touchscreen and transmitted from the GUI module 702 to the DSI driver module 710. The DSI driver module 710 then formats and transfers the video image data to the display driver 706 for presentation on the LDC touchscreen of The MD device.

In this exemplary embodiment, the sound measurement and capture functions of a typical electronic stethoscope are available as well with The MD device. The sound capture sensor may be a contact microphone 724 formed as a portion of the bell and diaphragm sensor on the exterior of The MD device. The contact microphone 724 contains an analog-to-digital (A/D) converter which translates the captured analog waveform from the contact microphone 724 and transmits the digitized sound data to the A/D chip driver 726 on the I2C communications bus 728. The digitized sound data is transmitted to the PHIMM 700 by the A/D chip driver 726. The PHIMM 700 may then direct the digitized sound data to be displayed to the medical practitioner in one of several formats based upon the modalities in operation when the sound measurement data is captured. The PHIMM 700 will always direct the heart rate processed from the incoming digitized sound data to be displayed on the LCD screen. A stethoscope application module 730 in communication with the PHIMM 700 transmits the heart rate data to the GUI module 702 with a command to format the heart rate data into the form the medical practitioner finds the most useful (character data, text data, or waveform data). The GUI module 702 then transmits the heart rate data to the touchscreen module 704 for display on the LCD touchscreen.

In this exemplary embodiment, the medical professional may have selected a modality in which an interpretation of heart sounds is selected. The PHIMM 700 may then direct the display of the heart rate data as well as directing the data to an auscultation analysis module 732 for an analysis of the heart sound data. The auscultation analysis module 732 will be engaged when a medical professional selects a modality for an analysis of any abnormal heart sound detected by the medical professional, or when the medical professional selects a modality for an analysis of an abnormal heart rhythm when an abnormal heart rhythm is detected by the medical professional. The auscultation analysis module 732 analyzes the incoming captured sound data and performs calculations to determine the type of abnormality that may be represented by the incoming sound data. Upon determining the interpretation of the heart sounds or heart rhythm, the auscultation analysis module 732 transmits this interpretation information to the GUI module 702. The GUI module 702 then directs the transmission of the analysis and interpretation data to be displayed to the medical professional on the LCD touchscreen by the touchscreen module 704.

In this exemplary embodiment, the medical practitioner may also select a modality for performing a pulse oximetry measurement. Upon selection, the pulse oximetry module 734 will communicate with the sensor to capture the slight difference in light level as the medical practitioner places the portion of The MD device containing the pulse oximetry sensor against a fingertip, earlobe or other body part. The pulse oximetry module 734 captures the measurement from the pulse oximetry sensor under the direction of the pulse oximetry chip driver 736, transmitting the captured sensor data using a digital Input/Output (I/O) driver 738 to transmit the captured measurement data to the pulse oximetry application module 740. The pulse oximetry application module 740 interprets the incoming pulse oximetry sensor data into a form that may be transmitted to the GUI module 702. The GUI module 702 will then direct the transmission of the formatted pulse oximetry data to the touchscreen module 704 for display to the medical professional on the LCD touchscreen display. Additionally, the pulse oximetry application module 740 may transmit the formatted data to the PHIMM 700 for storage in the internal digital memory of The MD device for later recall and use.

In an exemplary embodiment, the medical professional may also select a modality in which ultrasound measurement data is captured and displayed by The MD device. The PHIMM 700 may direct the GUI module 702 to display on the LCD touchscreen the modality selected as an ultrasound modality, and to direct the medical practitioner to connect the external ultrasound probe to the USB port dedicated to the ultrasound probe. The probe driver 742 may then become active, connecting with the ultrasound probe and communicating all measurement data from the probe to the USB bus driver 744 through the FPGA interface module 746 and directly to the ultrasound probe driver module 748. The ultrasound probe driver 748 maintains the operation and control of data to and from the ultrasound probe and transmits the incoming data to the ultrasound application module 750. The ultrasound application module 750 is operative to format the incoming ultrasound data into a format that may be displayed to a medical practitioner. The ultrasound application module 750 is also operative to receive selection and action commands from a medical practitioner as relayed from the touchscreen module 704 through the GUI module 702. The ultrasound application module 750 may then take a snapshot of the incoming ultrasound data and pass this data package to the PHIMM 700 for storage in the digital memory of The MD device. Alternatively, the medical practitioner may request that a segment of the ultrasound data be retained as a record. The ultrasound application module 750 may then format and transmit this section of the ultrasound measurement data to the PHIMM 700 for storage in the digital memory of The MD device, subject to the memory storage limitations of the digital memory.

In an alternative embodiment, a medical practitioner or medical student may request that portions of the measurement data recorded by any selected modality be recalled from the digital memory for export. The medical practitioner or medical student may send a command from the LCD touchscreen through the touchscreen module 704. The medical practitioner command is interpreted by the GUI module 702 and transmitted to the PHIMM 700. Upon receipt of the medical practitioner command, the PHIMM 700 retrieves the requested stored data from the integral digital memory for transmission to an external device. The PHIMM 700 transmits the recalled data to the file I/O module 752 for formatting and transmission to an external storage device. The external storage device may be contained within an external PC or other computer system, or may take the form of any other storage device such as a thumb drive, external hard drive, network computer, or any other storage device containing a file system capable of receiving and storing the data transmitted from The MD device. In an additional embodiment, the recalled data may be displayed on the LCD touchscreen device, or may be exported through the external mini-USB port to a router, network server, or other networked device to transmit the recalled data for TeleMedicine consultations or for referrals from other medical practitioners located at sites distant from The MD device.

In an alternative embodiment, data recalled from The MD digital memory may be transmitted to one or more external storage devices, computer processors, servers, networked devices, or any other device capable of accepting data communications through a wireless transmission element. The wireless transmission element may be a wireless transceiver installed within The MD apparatus and in data communication with the central processor of The MD. The wireless transceiver may be capable of utilizing any wireless transmission format, such as, by way of example and not of limitation, Wi-Fi, Bluetooth, Bluetooth Low Energy (BLE) or any other wireless protocol that may be built into the wireless transceiver and permit the transmission and reception of data communications over a wireless interconnection.

While certain illustrative embodiments have been described, it is evident that many alternatives, modifications, permutations and variations will become apparent to those skilled in the art in light of the foregoing description.

What is claimed is:

1. A medical diagnostic apparatus, comprising:
a processor, and electronic storage associated with said processor;
two or more sensor elements for measuring and capturing audio, video and multimedia data;
a screen display element;
a case enclosing the processor, electronic storage, all sensor elements, and the screen display element;
a plurality of selectable modalities with which a user may select the sensor element to be utilized to measure and capture audio, video, or multimedia data, or a combination of these types of data;
the audio, video, and multimedia sensor elements operative to present measured and captured sensor data to a user in real-time;
the electronic storage active to store the data from the audio, video, and multimedia sensor elements upon user direction.

2. The apparatus of claim 1, further comprising one or more audio transfer elements to permit one or more users to listen to the audio data as it is captured by an audio data measurement and capture sensor element.

3. The apparatus of claim 1, further comprising one or more data output devices operative to recall stored data from the electronic storage and transmit said recalled stored data to devices exterior to the medical diagnostic apparatus at the direction of the user.

4. The apparatus of claim 1, where the audio data captured by an audio sensor element may comprise bodily sounds from a patient who is placed in contact with the audio data sensor element.

5. The apparatus of claim 4, where the bodily sounds may comprise sounds made by the heart, lungs, within the abdomen, or within any other portion of a patient's body coming into contact with the audio data sensor element.

6. The apparatus of claim 1, where the audio sensor elements may comprise any one of a microphone, a bell and diaphragm, or any other sound sensitive audio pickup sensor.

7. The apparatus of claim 1, where the video sensor element may comprise a digital camera.

8. The apparatus of claim 1, where the multimedia sensor elements may comprise an ultrasound probe or a combination of a sound sensor element and said ultrasound probe.

9. The apparatus of claim 1, where the screen display element may further comprise a Liquid Crystal Diode touch enabled screen display.

10. The apparatus of claim 1, where user selectable modalities may be any one of sound capture, pulse oximetry, interpretation of heart sounds, interpretation of heart rhythm, ultrasound capture, video capture, or data transfer from the apparatus to an exterior device connected to the apparatus either through the use of a wire or wirelessly.
